(19) United States
(12) Patent Application Publication
(10) Pub. No.: US 2025/0099382 A1
(43) Pub. Date: Mar. 27, 2025

(54) Colloidal suspension of nutraceutical ingredients to treat traumatic brain injury

(71) Applicant: Robert Austin Chittenden, Round Lake, IL (US)

(72) Inventor: Robert Austin Chittenden, Round Lake, IL (US)

(21) Appl. No.: 18/373,068

(22) Filed: Sep. 26, 2023

(51) Int. Cl. A61K 9/127; A61K 31/05; A61K 31/12; A61K 31/198; A61K 31/221; A61K 31/352; A61K 31/7076; A61K 31/714; B82Y 5/00; B82Y 30/00

(52) U.S. Cl. CPC A61K 9/127; A61K 31/05; A61K 31/12; A61K 31/198; A61K 31/221; A61K 31/352; A61K 31/7076; A61K 31/714; B82Y 5/00; B82Y 30/00

(57) ABSTRACT

The product comprises a colloidal suspension which includes the ingredients liposomal adenosine triphosphate salt (liposomal ATP), methylcobalamin, glutathione, liposomal curcumin, liposomal resveratrol, taurine, fulvic acid, and acetyl L-carnitine, which will be delivered intravenously or by other parenteral means for the treatment of traumatic brain injury. Published studies indicate these substances, tested individually, reduce neuroinflammation or other secondary pathologies caused by a brain injury. Combination of the substances into a single intravenous colloidal suspension offers significant potential to directly reduce oxidative damage and directly improve the brain's energy crisis caused by traumatic brain injury (TBI), thereby mitigating the pathophysiological cascade of secondary damage after TBI. The product will be modified by removing glutathione for patients with a history of autoimmune disorders Stevens-Johnson syndrome or toxic epidermal necrolysis. Precise dosages of liposomal ATP, liposomal curcumin, liposomal resveratrol, taurine, and fulvic acid will be determined by various laboratory and medical tests explained in other sections of the application.

### BACKGROUND OF THE INVENTION

The Center for Disease Control reported 2.5 million traumatic brain injuries in the United States in 2010, and 2.8 million in 2013.¹ Some global estimates for traumatic brain injury (TBI) exceed 60 million cases.² Mass et al. report that 50 million people per year sustain a brain injury, and that yearly societal costs for TBI approximate U.S. $400 billion.³

TBI can cause a myriad of neurological and cognitive dysfunctions affecting memory, processing speed, logical and emotional processing, mental focus, coordination, brain circadian patterns, energy, and many other capacities. Initial injuries from TBI may include hematoma, diffuse axonal injury, neuronal necrosis, and vascular injury. A brain injury triggers a pathophysiological cascade of adverse molecular events, including neuronal efflux of potassium and neuronal influx of sodium causing depolarization and impaired neuronal communication, excess calcium accumulation in neurons, increased neuronal apoptosis, excess glycolysis due to mitochondrial damage and disrupted neuronal communication, lipid peroxidation, ischemia due to diminished blood supply to certain brain regions, blood-brain barrier disruption, circadian pattern disruption, cerebral edema, and other adverse effects that increase intracranial pressure and neuroinflammation.

According to the Mayo Clinic, the standard of care for most mild TBI cases is rest and analgesic medication for headache. For moderate or severe TBI, standard treatment includes medications to diminish inflammation, anti-diuretics to help reduce cerebral edema and intracranial pressure, anti-seizure medication, treating hemorrhage, increasing oxygenation, and possibly coma-inducing medication to decrease brain oxygen requirements.⁴ “Guidelines for the Management of Severe Traumatic Brain Injury”, 4^{th} Edition, published in 2016 by the Brain Trauma Foundation, American Association of Neurological Surgeons, and the Congress of Neurological Surgeons, offers detailed guidance for brain injury care similar to that of the Mayo Clinic.⁵

One study review indicated that standard analgesics are insufficient to prevent tissue damage and functional impairment.⁶ In addition, dexamethasone failed to maintain efficacy against inflammation after TBI.^{6,7} Thus, the current standard of care for TBI has failed to demonstrate strong efficacy in directly diminishing oxidative damage and the brain's energy crisis that contribute much to the secondary pathology caused by TBI.

Conventional treatment for moderate and severe TBI often includes general anesthetic medications, such as propofol, phenobarbital, or pentobarbital, to decrease brain oxygen requirements. Propofol also provides antioxidant capability by augmenting the Nrf2 antioxidant pathway.⁸ With respect to brain injury, a difficulty with these anesthetics is that they hyperpolarize neurons and glia by activating GABA_{A} receptors, which causes a significant influx of chloride ions into brain cells already dealing with excess sodium, chloride, and water causing cytotoxic edema.^{9,10,11} The added influx of chloride ions could further magnify the cytotoxic edema for these brain cells and increase lesion volume. For this reason, when TBI is treated with anesthetics, mannitol is typically also utilized to draw excess water from brain parenchyma to diminish cerebral edema and intracranial pressure. Although propofol does provide antioxidant benefits for TBI, because of the edema side-effects, it would be best to rely primarily on other treatments to directly mitigate oxidative stress, regardless of whether propofol is included in the treatment plan.

Any treatment plan for brain injury would necessarily include continual monitoring of patient progress. Such monitoring may include laboratory testing such as comprehensive metabolic panel, CBC, iron panel to rule out anemia, ELISA tests for inflammation, including IL-1b, IL-6, IL-8, TNF-a, NFkB, chemokine CCL2 which recruits macrophages after TBI, malondialdehyde to gauge lipid peroxidation, GSH redox, standard coagulation testing, von Willebrand factor, clotting factors 2a, 5, 10a, glial fibrillary acidic protein, ubiquitin carboxy-terminal hydrolase L1, neurofilament light, catecholamines, systolic blood pressure, mean arterial pressure, brain perfusion pressure, intracranial pressure, and brain tissue oxygenation (PtbO₂). Sodium and insulin levels are carefully monitored, since TBI disrupts metabolism of these substances. The “Neuroinflammation Panel” by NanoString Technologies would evaluate expression of 770 different genes relating to neuroinflammation, immunity, stress, metabolism, and neuropathology. The Montreal Cognitive Assessment is often administered at certain points in the patient's recovery.

To monitor hemostasis after TBI, standard tests include prothrombin time (PT), activated partial thromboplastin time (aPTT), D-dimer, and fibrinogen. The formula for calculating the international normalized ratio is INR=patient's result for prothrombin time/normal patient average. For prothrombin time, some laboratories utilize a reagent with thromboplastin and calcium chloride, and evaluate clotting time with a photo-optic device. A depletion of clotting factors, which some published studies indicate may occur after TBI, would correspond with increased clotting time.

Brain injury causes excess clotting and microthrombosis in more severe cases. It appears that for a significant number of more severe TBI cases, generally within the first day, excess coagulation depletes platelet factors and platelet hypofunction ensues. Subsequent decreased platelet counts also correlate with increased hemorrhage and mortality. Moderate or severe brain injury causes catecholamine excess which leads to hypertension and tachycardia for the first twelve hours or so after injury, followed by catecholamine depletion and systolic hypotension. Beta blockers such as propranolol are generally employed to diminish catecholamines for the first day. Thus, essential objectives of any treatment protocol include careful monitoring of the effects of treatment on hemostasis and catecholamine production.

Although conventional treatments and their respective dosages administered for preclinical studies could remain consistent since the injury administered to the study animals is identical for all animals, the treatment of traumatic brain injury in the clinical setting requires that conventional therapies be customized to the particular circumstances of a given patient's injuries. Any variability in treatment or drug dosages (i.e., analgesics, propofol, phenytoin, thiazides or mannitol, beta blockers, etc.) would likely remain somewhat evenly distributed between test groups and control groups, such that clinical results indicating efficacy for the test item treatment drug should retain statistical significance despite some variances in conventional treatments between patients in general. After conclusion of a clinical study, biostatisticians would verify that any variances in conventional treatments between test and control groups did not alter statistical significance for test group results indicating efficacy. One possible cause for conventional treatment variability between the test group and control group may result if the treatment drug significantly decreased the catecholamine depletion, such that somewhat fewer beta blockers were required for the test group. If a treatment test drug significantly decreased certain other symptoms for the test group, this could also translate into generally lesser dosages of certain conventional medications, compared to the control group. An essential point is that any new drug proposed to treat TBI will face precisely the same issues, since medical care for TBI will always remain prioritized over mere clinical testing.

### SUMMARY OF INVENTION

The product comprises a colloidal suspension of liposomal adenosine triphosphate salt (liposomal ATP), methylcobalamin, glutathione, liposomal curcumin, liposomal resveratrol, taurine, fulvic acid, and acetyl L-carnitine that will be delivered intravenously or by other parenteral means to treat traumatic brain injury. Several of the ingredients will be encapsulated in liposomes that will optimally range from 70 nanometers (nm) to 100 nm in diameter, which will reduce immune system clearance of the ingredients and attenuate degradation of the compounds prior to delivery to injured brain areas. Glutathione (GSH) will be removed from the product for patients with a history of Stevens-Johnson syndrome or toxic epidermal necrolysis, until additional testing can better determine whether GSH in any way adds to symptoms of these medical conditions. It is possible that adverse effects from GSH administration for patients with these autoimmune disorders will only occur if such administration causes excess blood levels of GSH, which may not occur with more severe cases of TBI, where GSH tends to be depleted. Precise dosages of liposomal ATP, liposomal curcumin, liposomal resveratrol, taurine, and fulvic acid will be determined by various laboratory tests delineated in other sections of the application.

The current standard of medical treatment for traumatic brain injury provides few effective interventions to directly attenuate oxidative damage to neurons, and no treatments to directly improve the brain's energy status, even though the present standard of care for TBI does indirectly accomplish these objectives to some degree. Treatments that deal directly with these objectives and that also bring minimal potential toxicities will provide an increased quality of medical care for traumatic brain injury.

This invention offers significant potential to directly decrease oxidative damage, including lipid peroxidation, to neurons and glia, decreasing neuroinflammation, and to directly improve the brain's energy status and resultant neuro-communication, reversing damage to the blood-brain barrier, and reducing cerebral edema and intracranial pressure. Clinical progress against these pathologies secondary to the original brain injury would improve patient recovery.

### DESCRIPTION OF INVENTION

The product is a colloidal suspension comprised of multiple ingredients dispersed in a suitable dispersion medium. Ingredients include liposomal adenosine triphosphate salt, methylcobalamin, glutathione, liposomal curcumin, liposomal resveratrol, taurine, fulvic acid, and acetyl L-carnitine. Encapsulation of several of the ingredients into liposomes should increase delivery of the substances across the blood-brain barrier. Liposomes will generally be less than 120 nanometers in diameter, and optimally between 70 nm and 100 nm in diameter, to decrease immune system clearance and facilitate improved delivery across the blood-brain barrier and into neurons.¹² Liposomes used to encapsulate ingredients will generally have a neutral surface charge, since positive (cationic) surface charge liposomes are toxic, due to interaction with negatively charged (anionic) molecules in blood.¹³ Endothelial cells that line the luminal interior of brain capillaries that form the blood-brain barrier are negatively charged, and would thus tend to repel liposomes with an anionic surface charge. One skilled in the art could purchase the individual ingredients of this invention, pre-manufactured, from suppliers, and would probably find this to be a more economical means of producing the final invention. However, instruction is still provided for how one would manufacture the ingredients. All ingredients included in the product are generally recognized as nontoxic. Later in the description, nontoxicity for the ingredients will be explained, in addition to the method for determining preliminary dosages for clinical studies for certain ingredients of the product. When the terms “IV product”, “IV products”, “the formula”, or “IV composite formula” are used, these terms refer to the products of this invention that may be delivered by any parenteral means.

Since this invention will serve as a medical intervention, it must be manufactured aseptically. To ensure aseptic manufacturing, one is advised to consult the U.S. FDA's publication, “Guidance for Industry—Sterile Drug Products Produced by Aseptic Processing—Current Good Manufacturing Practice”.¹⁴ According to this document, microfiltration with pores of 200 nanometers or less is generally adequate to remove pathogens. General guidelines are provided for microbial testing to validate filtration efficiency. Since 2001, most pharmaceutical companies internationally converted to aseptic standards developed by the International Organization for Standardization (ISO).

Adenosine triphosphate magnesium, C₁₀H₁₆N₅O₁₃P₃×Mg₂, an ATP salt, is produced by phosphorylating adenosine diphosphate with a phosphate group. ATP is the main currency of energy in living cells. The molecule is comprised of the nucleobase adenine attached to a ribose moiety, which is in turn bonded with three phosphate groups The energy retained in ATP's triphosphate chain causes the three phosphate groups to remain connected despite the negative charge for each of the phosphates. Cationic magnesium ions bond with ATP at the oxygen atoms of the phosphates, which helps stabilize the molecule

ATP is a vital factor for maintaining proper cellular ion levels necessary for communication between brain cells. To facilitate this, integrated into the membranes of brain cells (and all living cells) are molecular transporters that shuttle ions across membranes against their concentration gradients to maintain proper intracellular ion levels necessary to sustain life. Transmembrane sodium-potassium ATPase enzymes utilize the energy from one ATP molecule to transport three sodium ions out of the cell and two potassium ions into the cell. The transmembrane enzyme plasma membrane calcium ATPase relies upon ATP to export excess calcium out of the cell. After a brain injury, damage to neuron membranes and subsequent excess extracellular glutamate cause an excessive neuronal loss of potassium and accumulation of too much calcium in neurons. As a result, the brain is often unable to produce sufficient energy (ATP) through glycolysis to sufficiently energize the ATPase enzymes that must work to restore proper potassium levels in cells and to clear excess calcium. Insufficient brain energy after TBI impairs neuronal communication and often leads to increased neuronal apoptosis through the intracellular surplus of Ca²⁺and consequent escalation of caspase 3. Excess neuronal calcium also triggers calpain 2, which breaks down cellular architecture, an additional route for neural apoptosis, the demise of neurons. Thus, an objective of this invention is to provide a direct source of additional ATP to help enzyme systems restore optimal ion levels in neurons, which in turn will mitigate signaling for neuroinflammation and neuronal apoptosis. Yet there are obstacles to this process that must be worked through carefully.

After a traumatic brain injury, ATP migrates out of damaged neurons and glia into the cerebral extracellular space, which escalates microglia activation, cytokine production, and consequent neuroinflammation. ATP is thus a major signaling molecule for neuroinflammation after TBI. Some researchers are considering blocking extracellular ATP (eATP) signaling after TBI as a means to diminish eATP's damaging pro-inflammatory effects.¹⁵ At the same time, several other studies indicate that ATP encapsulated in liposomes mitigates neuroinflammation and demise of neurons caused by ischemia.^{16,17,18,19} This indicates that encapsulation of ATP in certain types of liposomes delivers ATP across the blood-brain barrier and brain cell membranes without dispensing ATP into the brain's extracellular space. Thus, it appears that liposomal ATP would effectively and directly mitigate the brain's energy crisis and oxidative damage that otherwise would occur after TBI. Cerebral microdialysis will be utilized to determine whether administration of liposomal ATP avoids increasing extracellular ATP after injury. Falzoni et al. describe a recent innovation to evaluate levels of extracellular ATP based upon an engineered protein.²⁰ If eATP remains relatively unchanged after administration of IV liposomal ATP, this will indicate that liposomal ATP is being delivered across the blood-brain barrier and into neuron and glia membranes to help restore proper intracellular potassium and calcium levels. The level of cholesterol incorporated into liposomal membranes may be adjusted to decrease any dispensing of ATP from liposomes prior to delivery to brain cell membranes.

There are multiple processes for producing ATP, prior to encapsulation of ATP in liposomes. Yan et al., describe a method of producing ATP utilizing adenosine and sodium hexametaphosphate as the main reactants working with artificial scaffolding proteins based upon CBM3 (carbohydrate binding module 3), certain immunity proteins and covalent labeling proteins.²¹ Caton-Williams and colleagues delineate a system for manufacturing ATP in which salicyl phosphorochloridite is dissolved in dimethylformamide (miscible in water) and tributylamine, and then is reacted with pyrophosphate. Adenosine is then added and mixed at 25° C. for 90 minutes. Next, molecular iodine, pyrimidine, and water are added to yield adenosine triphosphates. Chromatography is then employed to separate the ATP from reaction byproducts and any toxic residues of the solvents.²² Magnesium stabilizes the ATP molecule. ATP salts are subsequently encapsulated into liposomes.

Methylcobalamin, methylated vitamin B12, is a complex molecule based on cobalt, with a methyl group attached to cobalt instead of a cyano group as with cyanocobalamin. According to the Cleveland Clinic, as many as 15% of the general population is deficient in vitamin B12.²³ A deficiency in methylated B12 (methylcobalamin) may diminish methylation, or may cause forms of anemia, where aberrations in formation of new red blood cells reduce the supply of functional erythrocytes capable of transporting oxygen and nutrients. After a brain injury, it will be imperative to correct potential anemia to optimize erythrocyte function. Preclinical studies indicate that methylcobalamin improves recovery of axons by attenuating endoplasmic reticulum stress and consequent neuronal apoptosis.²⁴

Methylcobalamin can be manufactured through multiple processes. Microbial fermentation is preferable to purely chemical synthesis, given the many steps in cobalamin synthesis. Two microbial strains often employed in microbial fermentation are Propionibacterium freudenreichii and Pseudomonas denitrificans. P. freudenreichii is generally recognized as safe by the U.S. FDA. This further diminishes potential toxicities, a primary objective for any medication. Calvillo et al. describe several processes for manufacture of cobalamin that utilize P. freudenreichii (Nutra Strategic Solutions, Inc.), a source of carbon such as glucose or maltose syrup, tryptone (Sigma-Aldrich) for source of peptides and nitrogen, and other nutrients such as magnesium sulfate, that are mixed in a bioreactor (e.g., Sartorius Biostat STR Microbial, which is GMP compliant).²⁵ Glucose sources are autoclaved for sterilization. In one process, the mixture is fermented anaerobically in a bioreactor, with N₂ gas to help maintain anaerobic conditions. Light agitation is maintained throughout the process to reduce settling out of any components. To augment cobalamin production, at 96 hours, 5,6-dimethyl-benzimidazole (DMBI) is added and nitrogen gas delivery to the bioreactor is stopped to allow some oxygen, albeit reduced, to the fermentation mixture. Cyanocobalamin can be reduced with reducing agents such as lithium borohydride or sodium borohydride, and then methylated with a methylating agent such as trimethylsulfonium iodide. Cobalt chloride may be used to remove cyanide ions. The dosages of methylcobalamin included in the IV products will be similar to dosages used in conventional supplementation, where general non-toxicity is very well established.²⁶

Glutathione, C₁₀H₁₇N₃O₆S, is a tripeptide of amino acids cysteine, glutamic acid, and glycine, with a thiol group from cysteine. In reduced form, glutathione neutralizes oxidation from free radicals. A greater percentage of oxidized glutathione to reduced glutathione reflects increased oxidative stress. Iron status must be carefully monitored after brain injury. Iron is necessary to carry oxygen in the blood for delivery to cells, including injured neurons and glia, for energy production in mitochondria. Microbleeding into brain parenchyma after TBI often dysregulates cerebral iron metabolism, causing excess iron to accumulate in neurons, which augments production of reactive oxygen species and oxidative stress, in turn magnifying damaging neuroinflammation. Glutathione deficiency generally correlates with excess cerebral free iron, ROS production, and ferroptosis. Glutathione binds with intracellular surplus free iron, which neutralizes the pro-oxidation effects of the metal, thereby mitigating its pro-inflammatory influence. Glutathione levels (in reduced form) are often diminished during the first four to five days after brain injury, impairing the brain's ability to neutralize surplus iron in neurons and glia.²⁷ A primary objective for including glutathione in the IV formula is to neutralize the oxidative damage caused by surplus iron. Cerebral microdialysis will be utilized to monitor iron levels in brain extracellular space, which approximates intracellular iron status.

Glutathione, C₁₀H₁₇N₃O₆S, is biosynthesized from amino acids, glutamate, cysteine, and glycine. L-glutamate and cysteine are reacted with enzyme gamma-glutamyl cysteine synthase to yield gamma-glutamylcysteine. This intermediate compound is reacted with glycine via enzyme glutathione synthase to produce glutathione. A common vehicle for the biosynthesis of glutathione (GSH) is Saccharomyces cerevisiae yeast, which is often genetically modified to augment expression of genes for production of the above enzymes, which in turn amplifies GSH content. GSH can be produced from S. cerevisiae by mixing malt extract, soybean peptone, glucose, yeast extract, sucrose, MgSO₄, (NH₄)₂HPO₄, and K₂HPO₄, with pH at 5.8, at 30° C. for 72 hours, with agitation at 200 rpm, in a bioreactor.²⁸ GSH content of the yeast can be extracted with microfiltration and nanofiltration. Wang et al. describe a method to extract GSH from yeast mixing yeast and water at 1:10 ratio at 78° C., with agitation for 10 minutes. The resulting suspension is ultrafiltered to remove large molecules, then nanofiltered for GSH.²⁹ U.S. Pat. No. 8,188,308B2 provides an excellent summary of various methodologies to produce GSH. Elements of delineated processes for GSH generally include a microbial vehicle capable of producing GSH, a carbon source, nitrogen source, mineral and vitamin sources, 20° C. to 50° C., pH 6 to 7.5, culturing time 5 hours to 5 days.

Glutathione will be removed from the formula for patients with a history of Stevens-Johnson Syndrome (SJS) or toxic epidermal necrolysis, until additional medical and genetic testing determines whether glutathione (GSH) adds to symptoms of these dermatological autoimmune disorders. Some reports have indicated that these conditions may be adversely affected by glutathione. These rare autoimmune conditions can be triggered by certain medications, including sulfa drugs and anticonvulsants such as phenytoin or Lamictal. Symptoms of SJS include pyrexia, fatigue, erythema, blisters, and headache. SJS and toxic epidermal necrolysis are differentiated mainly in terms of the severity of symptoms. It is possible that in the context of brain injury where GSH levels are depleted, that supplemental GSH would merely replenish deficient stores of GSH without producing excess blood levels, and that IV GSH could only worsen SJS if the GSH were to increase blood GSH to excessive levels. If a TBI patient's GSH levels are only somewhat diminished, it may be possible to administer intravenous GSH separately at a less than standard dosage that would merely bring the patient's GSH to optimal levels, and that this could avoid triggering any adverse symptoms.

One possible method of medical treatment for the patient with SJS and TBI would be for the physician to evaluate the patient's glutathione status, and then determine whether to administer a reduced dosage of IV GSH separately from the IV composite formula after obtaining the patient's fully informed consent. One possible treatment objective would be to normalize glutathione status that had been determined to be deficient, while avoiding any symptoms of SJS Labcorp lists the reference range for blood test of GSH as 176 to 323 ug/ml (microgram per milliliter).³⁰ If the patient's GSH result were low, the physician would administer IV GSH at a reduced dosage compared to the dosage of GSH generally included in the IV composite formula. After each IV GSH administration, the patient's GSH would be monitored with either blood testing or other diagnostic means. The physician would know how to evaluate a patient's GSH status, how to modify dosages of IV GSH to normalize GSH levels, and how to evaluate for possible symptoms of SJS or toxic epidermal necrolysis.

Curcumin, C₂₁H₂₀O₆, the component of turmeric that provides its yellow pigmentation, is a polyphenol compound with one phenol group attached on either side of a pair of carbonyl groups. Curcumin is extracted from turmeric root and is commonly used for culinary and medicinal purposes. Curcumin has strong anti-inflammatory, immunomodulatory, antithrombotic, and neuroprotective properties.³¹⁻³⁶ This compound exerts numerous anti-inflammatory effects by diminishing NFkB's transcription of genes for pro-inflammatory cytokines and chemokines.

Curcumin is commonly separated from turmeric with solvent extraction and chromatography.³⁷ Ethanol is often used for the solvent, at a temperature of 60° C. to 80° C. A slightly different methodology, supercritical fluid extraction, utilizes carbon dioxide as the solvent in supercritical conditions.³⁸ First, an extraction chamber is filled with ground turmeric rhizomes. Next, a pump forces pressurized CO₂ in supercritical state into the extraction chamber. In the chamber, the supercritical CO₂ dissolves the curcuminoids and oleoresins. Next, the supercritical CO₂ carries the dissolved curcuminoids past a pressure release valve into a separator container. In the separator, the pressure is lower, and the CO₂ separates from the curcuminoids, which are then collected from the separator (high pressure reactor from Sitec Sieber, https://www.deheerbv.com). Encapsulation of curcumin in neutral charged liposomes would augment absorption. Curcumin is fat soluble, and thus will tend to incorporate within the interior of phosphatidylcholine liposomal membranes.

Some published scientific studies indicate that curcumin may decrease platelet aggregation.³⁹⁻⁴¹ This might be a difficulty after a brain injury, since platelet hypofunction is common after the first day following TBI, according to Kutcher et al.⁴² The study by Shah (reference #39) reports that curcumin reduced the production of thromboxane A2 by platelets and other factors. Kutcher explains that after more severe TBI, arachidonic acid levels decline significantly after 24 hours, which in turn causes a depletion of thromboxane A2, the same compound that is diminished by higher doses of curcumin. Thus, an important objective of curcumin administration for TBI is to find the optimal dosage that attenuates oxidative damage and other secondary pathologies, as is clearly indicated by published studies, yet avoids significant decreases in production of thromboxane A2 and beneficial amounts of platelet aggregation. Thromboxane A2 and its metabolite thromboxane B2 can be evaluated with standard laboratory testing, following varying dosages of liposomal curcumin.⁴³

Resveratrol, C₁₄H₁₂O₃, with chemical name 3,5,4′-Trihydroxystilbene, is a plant polyphenol with two phenol groups connected by a double styrene bond. This compound is generally found in grapes, cocoa, cranberries, blueberries, Japanese knotweed, and other fruits. Japanese knotweed (Polygonum cuspidatum) is probably the most abundant plant source for resveratrol. The trans-resveratrol isomer demonstrates greater antioxidant and anti-inflammatory properties compared to the cis isomer, and in scientific studies has demonstrated efficacy in mitigating secondary pathology from brain injury.⁴⁵⁻⁵² Resveratrol crosses the blood-brain barrier.

Resveratrol may be extracted from Japanese knotweed through conventional extraction utilizing a distilling flask, or via supercritical fluid extraction with carbon dioxide in supercritical conditions Wang et al. describe a system for isolating resveratrol from Japanese knotweed.⁵³ The roots of the plants are ground into a powder and placed in the distilling flask or vat with ethanol, approximately 6:1 ethanol to Japanese knotweed powder. Resveratrol has poor water solubility yet is soluble in ethanol. The mixture is allowed to sit for 12 hours at room temperature. Separation of resveratrol is furthered by reflux extraction at 80° C. Extract solutions are combined and evaporated in a rotary evaporator at 65° C. HPLC (high performance liquid chromatography) is then used to identify resveratrol in the extract, and to quantify the amount. Allowing the ethanol/Japanese knotweed mixture to steep for 72 hours or so in stainless steel vats should augment extraction of resveratrol from the herb. In addition, increasing the herb per ethanol ratio should increase the amount of resveratrol separated from Japanese knotweed by ethanol Resveratrol should be encapsulated into liposomes to improve bioavailability.

Turkez et al., report that resveratrol diminishes genetic damage induced by permethrin in chromosome tests with human lymphocytes in culture.⁵⁴ Resveratrol is generally nontoxic. However, published research studies indicate that resveratrol at certain dosages may reduce platelet aggregation.⁵⁵⁻⁵⁶ This might create difficulties since TBI after the first day may also cause platelet hypofunction. Laboratory testing will seek to determine optimal dosages of resveratrol that provide efficacy in treatment of TBI, which is indicated by published research, and that avoid interfering with necessary platelet aggregation. Varied dosages of resveratrol will be administered, followed by evaluation of various clotting factors, including prothrombin time. The maximum dosage that avoids diminishing platelet aggregation should fall within a range that also attains efficacy against secondary pathologies from TBI, since preclinical studies indicate resveratrol mitigates brain edema and neuroinflammation after TBI.

The amino acid taurine, C₂H₇NO₃S, provides strong anti-inflammatory benefits, diminishing multiple cytokines after brain injury, mitigating endoplasmic reticulum stress and surplus intraneuronal calcium, according to preclinical studies.⁵⁷⁻⁵⁹ Taurine is biosynthesized in the central nervous system through the cysteine sulfinate decarboxylase pathway. Taurine crosses the blood-brain barrier via the taurine transporter and is nontoxic. Taurine will generally not be contraindicated for any medical conditions, provided that certain higher dosages do not activate GABA_{A} receptors after TBI in a way that is statistically significant.

Medical or laboratory testing will be conducted to determine the maximum dosage of taurine that does not activate GABA_{A} receptors, which could cause excess migration of chloride ions and water into neurons already dealing will excess sodium, chloride, and cytotoxic edema. Diffusion tensor imaging is a system of radiological analysis that works with magnetic resonance imaging (MRI), and may be used to detect changes in intraneuronal water content after administration of taurine. Cerebral cytotoxic edema correlates with increased fractional anisotropy and reductions in mean diffusivity. For preclinical evaluations, patch clamp testing is commonly employed to analyze how compounds affect movement of ions across cell membranes. With the patch clamp procedure, brain tissue is removed from the animals and prepared with special solutions for examination. The micropipette is attached to the cell membrane, with an electrode inside the pipette. Test solution is administered through the pipette to the cell membrane, and effects on ion channels are recorded (www.moleculardevices.com). Scientists skilled in the art will know how to implement all such procedures.

Taurine, C₂H₇NO₃S, is a nonproteinogenic amino acid, where the sulfur atom connects with double bonds to each of two oxygen atoms, and with a single bond to a hydroxyl, and single bond to a carbon which in turn is covalently bonded to the remainder of the molecule. Ma et al. delineate a system to manufacture taurine.⁶⁰ Monoethanolamine (Fisher Scientific) is reacted with sulfuric acid in a bioreactor, which yields 2-aminoethyl hydrogen sulfate. This intermediate compound is reacted with sodium sulfite to yield taurine. With U.S. provisional patent application 2014/0121405, Chen describes a process to produce taurine from sodium taurate solution, ethylene oxide, and ammonia, which the inventor claims is less expensive, more efficient, and reduces pollution. Since taurine is commonly found in many foods, the amino acid is generally recognized as nontoxic

Fulvic acid, C₁₄H₁₂O₈, is an aromatic hydrocarbon with three aromatic rings and four hydroxyls. The compound is produced in soil from the decomposition of organic material by microbes. Cationic minerals such as zinc, manganese, iron, or copper bind to fulvic acid at the negatively charged hydroxyls. In this way, fulvic acid augments absorption of nutrients, which should improve patient recovery. Microhemorrhage after TBI often causes an accumulation of excess iron in brain parenchyma. Tests, including cerebral microdialysis (CMD), will be conducted to verify that fulvic acid works to maintain proper iron regulation, reducing iron toxicity proximate to damaged parenchyma. If study results from CMD indicate a particular dosage of fulvic acid increases iron levels in the brain extracellular space to toxic levels, dosages of fulvic acid will be adjusted accordingly to attenuate iron levels. Micro-dialysates will be evaluated with graphite furnace atomic absorption spectrometry (spectrometer from Thermo Fisher, www.thermofisher.com). The analyte sample is atomized in the graphite furnace, which breaks the chemical bonds of the various substances. The resulting elements absorb light with electromagnetic energies specific to particular elements and that correlate with combinations of specific frequencies on the electromagnetic spectrum. Computer algorithms record and interpret the results and report a qualitative and quantitative delineation of the analyte. Standard blood tests are employed to manage possible anemia.

Fulvic acid, C₁₄H₁₂O₈, may be produced by decomposing organic vegetation with fungi and/or probiotic strains produced through fermentation of vegetation. Van Dyke and colleagues describe a process to produce fulvic acid which they claim is more economical and efficient, and is delineated in U.S. provisional patent application 2012/0279266. Decomposed vegetation, i.e., compost and a crop, is heated and mixed with filtered water and blended into an aqueous mixture. Centrifugation is used to separate and remove solid material from mixture. The resultant liquid should contain perhaps 4% fulvic acid, which could be verified with spectrophotometric analysis (www.biocompare.com for spectrophotometers). Additional centrifugation may improve the percentage of fulvic acid in the product. Impurities could be removed with microfiltration or nanofiltration, and hydrochloric acid would help eradicate any remaining pathogens. Since fulvic acid reacts with ozone to form other carbonyl substances, ozone is not an option for sterilization. Temperatures exceeding 70° C. alter the molecular structure of fulvic acid in addition to its properties. This rules out steam sterilization which relies upon temperatures of 121° C. Fulvic acid is nontoxic and non-genotoxic.⁶¹

Acetyl L-carnitine (ALC), a quaternary amine, augments energy production from fatty acids by providing carnitine, which shuttles fatty acids across the mitochondrial inner membrane to be broken down through beta oxidation, a four-step process involving oxidation by flavin adenine dinucleotide and nicotinamide adenine dinucleotide. This process produces acetyl coenzyme A, which is then transferred to the citrus acid cycle, where the molecule is prepared for oxidative phosphorylation and consequent endothermic production of adenosine triphosphate (ATP) via the assembly line of ATP synthase. The acetyl moiety from ALC is donated as an acetyl group to choline, resulting in acetylcholine, which is facilitated by the enzyme choline acetyl transferase. ALC is an ester of L-carnitine, with acetylation synthesized by ALC-transferase. Additional carnitine, to support energy production in brain cells experiencing an energy crisis after TBI, and acetylcholine to aid neural communication, should improve recovery from brain injury. ALC crosses the blood-brain barrier and diminishes excess cortisol levels.⁶² This might permit some modifications to dosages of beta blockers often utilized for TBI treatment.

Acetyl L-carnitine, C₉H₁₇NO₄, may be manufactured by first producing carnitine, then adding an acetyl group. One means of production for carnitine, described by Nakayama and colleagues in U.S. Pat. No. 5,041,375, is to hydrolyze carnitine nitrile with nitrilase enzymes from aerobic, gram-positive, coryneform bacteria strains, to produce carnitine. The bacteria strains are cultivated with a hydrocarbon source, nitrogen source, minerals such as magnesium sulfate, and other nutrients at 20° C. to 40° C. in a bioreactor for one to seven days, then separated via centrifugation or filtration from the fermentation medium. The microbial cells with nitrilase are mixed with substrate carnitine nitrile in a bioreactor with moderate conditions for 1 to 6 days. Thin layer chromatography may be used to evaluate carnitine production. The resulting carnitine may be reacted with an acetyl group by mixing the carnitine with acetic acid, which yields acetyl L-carnitine, according to Kramer in U.S. Pat. No. 9,573,883. Since ALC is an endogenous substance with acetylation of L-carnitine occurring in the mitochondrial matrix, ALC is generally nontoxic.

Several of the formula ingredients will be encapsulated in neutral-charged liposomes with a dimension of less than 120 nanometers (nm) to reduce opsonization and immune clearance. Liposomes will be phosphatidylcholine diglycerides with saturated fats attached to the glycerol moiety, which compared to unsaturated fats, provide the liposome membranes with better stability, reduced subsequent permeability to exogenous substances, and reduced loss of encapsulated liposomal ingredients prior to delivery to brain parenchyma. Hydrocarbons with carbon double bonds tend to form angled structures at the double bond, which is why phospholipids with unsaturated fats do not align with adjacent similar molecules as compactly as phospholipids with saturated fat moieties. Cholesterol further augments liposome membrane stability, and will be incorporated into the membrane interior. Liposomal phospholipids may include dimyristoyl phosphatidylcholine, dipalmitoyl phosphatidylcholine, and distearoylphosphatidylcholine.

A common means of manufacturing liposomes for encapsulation of pharmaceutical ingredients is thin film hydration, followed by extrusion to reduce variances in liposome dimension and lamellarity. Phospholipids are first dissolved in an organic solvent, often ethanol. The ethanol is then evaporated from the mixture at approximately 50° C., which leaves a thin film of liposomes in the evaporation flask. A rotary evaporator may be utilized for this process (www.rotovaps.net). The phospholipids are then rehydrated with distilled water or saline buffer, at temperature above phase transition (Tₘ), so that rehydration occurs with lipids in liquid-crystalline phase. Moderate stirring may improve uniform dimension and distribution of liposomes in suspension. Liposome mixture is then cooled at 4° C. for 24 hours. To maximize the percentage of liposomes in the 70 nm to 100 nm range, the liposomal suspension may be nano-filtered through polycarbonate filters (www.genizer.com). Dynamic light scattering (DLS) may be employed to evaluate dispersity of liposome suspension (Wyatt Technology, www.wyatt.com).

Curcumin and resveratrol are both fat-soluble and will dissolve in ethanol. Thus, when preparing liposomes with thin film hydration, these two ingredients should be added to the phospholipid mixture together with the ethanol solvent. ATP salt is water-soluble and should therefore be added to the liposomal mixture during rehydration with distilled water or aqueous solution. Sturm et al. describe other methods for liposome preparation, which provide a degree of variability in factors such as amount of solvent required, encapsulation efficiency for ingredients, amount of time required, degree of difficulty in removing solvent, and other production factors.⁶³ For example, the proliposome method yields better encapsulation efficiency compared to some liposome preparation processes. Phospholipids are dissolved in ethanol and water, then heated to 60° C. for ten minutes and stirred to produce a paste. The paste is then allowed to cool. Water is very gradually added to the phospholipid mixture for an hour. The mixture is then sonicated and refrigerated for several days at 4° C. For this liposome process, curcumin and resveratrol would be added to the mixture with the ethanol, and ATP salt would be added with water during the gradual hydration phase.

Dvoriantchikova et al. describe a process for liposome preparation that facilitated exceptional preclinical results with liposomal ATP in mitigating inflammation and necrosis after ischemia-reperfusion injury.⁶⁴ Liposomes comprising either phosphatidylcholine (PC) or PC and phosphatidylserine at a 7 to 3 molar ratio were dissolved in chloroform, which was subsequently evaporated in a rotary evaporator. The resulting lipids were dried in a desiccator for 60 minutes, and then diffused in a vortex mixer with phosphate buffer solution. The suspension was then sonicated and centrifuged. This is similar to other liposome preparation methods. Ethanol could be used for the solvent instead of chloroform, since ethanol is less toxic. Extrusion through a polycarbonate membrane filter could be employed to maximize liposomes in the 70 nm to 100 nm range.

After liposome preparation and ingredient encapsulation processes are completed, the remaining non-liposomal ingredients may be added to the suspension mixture. At this time, a certain percentage of negatively charged phospholipids, such as phosphatidylserine or phosphatidylinositol, may be added to the suspension mixture to augment electrostatic repulsion throughout the suspension which will improve colloidal stability. All ingredients could be mixed in an industrial mixer (www.vmimixing.com).

Statistical analysis will be utilized to determine if results from separate studies on liposomal ATP, liposomal curcumin, liposomal resveratrol, taurine, and fulvic acid, in addition to all other preclinical and clinical studies, are statistically significant. MS Excel can be used to calculate the analysis of variance (ANOVA) for study results. Generally, 5% is used as the alpha value, such that if calculations indicate there is a less than five percent chance that the null hypothesis is true when the results indicate otherwise, the study results are deemed to be statistically significant. Preclinical studies will include eight animals per group. Analysis of results will be based upon the t distribution with seven degrees of freedom. If clinical studies enroll thirty or more participants for each group, analysis of results will follow the normal distribution or Z distribution. This means that for the Z distribution a slightly lesser variance in test results between test groups and control groups could create statistical significance, compared to the t distribution for samples of less than thirty. For clinical studies, once statistical significance is established, the physicians must then decide if the study results are clinically significant in terms of providing substantial medical benefits to the study participants.

All ingredients of the product are generally nontoxic. Evidence from published research also indicates the ingredients, tested individually, provide efficacy in the treatment of TBI, mitigating secondary pathology after the original injury. Much of the research for toxicity and efficacy studies is preclinical, which necessitates a conversion of preclinical data to human equivalent data for interpretation of implications of preclinical data to clinical studies. Nair et al. explain dosage conversion criteria used to translate preclinical data into human clinical equivalent data for the purpose of setting preliminary dosage regimens for clinical studies.⁶⁵ Rodents have much quicker metabolisms than humans. The average heart rate for mice is 500 to 700 beats per minute. This means that rodents will metabolize substances much more quickly than humans, which necessitates higher dosages per kilogram of subject weight to achieve similar metabolic effects. Although humans have greater weight than rodents, the animals' quicker metabolism partially offsets the weight variable for the purpose of determining dosage equivalents between species. Nair et al. delineate conversion factors between humans and various other species. For conversion of preclinical data from mice to human, divide the mg/kg dosage by 12.3. One would then multiply the result by an approximated average weight in kilograms for human clinical subjects to establish a preliminary clinical dosage. For example, if the preclinical dosage indicating drug efficacy were 100 mg/kg, this dosage would be divided by 12.3 to get 8.13 mg/kg, which would then be multiplied by an average weight in kilograms designated for the clinical study. For an average clinical subject weight of 65 kilograms, the original 100 mg/kg preclinical dosage would translate to 528 mg, which would be the preliminary dosage for clinical studies. For conversion of preclinical data from rat to human, the animal dosage would be divided by 6.2, then multiplied by the average subject weight in kilograms for the clinical study.

According to PubChem.gov (https://pubchem.ncbi.nlm.nih.gov/), a website of the National Institutes of Health, adenosine triphosphate salt (ATP) is generally nontoxic, with an LD50 of greater than 2 grams per kilogram of animal weight. In addition to being nontoxic in proper amounts, ATP is indeed essential for life. In the context of TBI where intraneuronal ATP is seriously depleted, an exogenous source of ATP delivered directly to neurons should preserve tissue. Utilizing Nair's conversion criterion for the LD50, (greater than) 2 grams divided by 6.2 (for rat), times 65 kilograms, is about (greater than) 21 grams of ATP for human equivalent data. The IV composite formula dosage for liposomal ATP will provide a dosage that exceeds 10 mg, yet will be nowhere near 21 grams. An important point is that LD50 studies with ATP typically do not involve liposomal encapsulated ATP, which means that for such studies, all ATP is delivered into the plasma and possibly the extracellular space, which at a certain point will signal for increased inflammation. One research study with ATP used a dosage of 400 mg per day, yet the ATP was not in liposomal form.⁶⁶ The optimal dosage for ATP encapsulated in liposomes that provides efficacy against TBI while avoiding increases in cerebral extracellular ATP should exceed 10 mg, which is why this amount is selected as the lower dosage parameter for ATP for the IV formula. Studies regarding pharmacokinetics, pharmacodynamics, general toxicity, genotoxicity, efficacy in diminishing TBI pathologies, and effects on extracellular ATP and adrenergic signaling will guide determination of optimal formula dosages for liposomal ATP.

Methylcobalamin is found in many foods, and in higher doses in many nutritional supplements. General nontoxicity is well established. The Mayo Clinic lists vitamin B12 as “cobalamin”, which would include methylcobalamin, cyanocobalamin, and other variants, and recognizes cobalamin as generally safe even at higher doses.⁶⁷ Although the common forms of cobalamin are considered nontoxic, methylated cobalamin is still a better form of the vitamin for people with genetic deficiencies in methylation. Methylcobalamin is preferred to cyanocobalamin, since at higher doses cyanocobalamin may result in additional cyanide molecules finding their way to damaged tissue, which may present some difficulties in the context of brain injury.

Glutathione (GSH) is an endogenous compound necessary for proper liver function and good health. Accordingly, in proper amounts GSH is nontoxic. PubChem.gov indicates that intravenous GSH produced an LD50 of 2.2 grams per kilogram of animal weight. To convert this preclinical data to human equivalent data, 2.2 grams divided by 12.3 is 179 mg, times 65 kilograms for a human subject weighing 65 kg is approximately 11.6 grams. However, an important factor to consider is that after a traumatic brain injury, the requirements for GSH in reduced form are greatly increased. This suggests that an LD50 test for GSH in the context of TBI as opposed to an LD50 with otherwise healthy animals may produce results significantly greater than the 2.2 grams per kilogram result reported at PubChem.gov Inclusion of GSH in the IV composite formula will seek to restore and maintain optimal GSH levels and optimal cerebral free iron levels.

For curcumin, PubChem.gov reports an LD50 of greater than 2 grams per kilogram of animal weight, which reflects a human equivalent of approximately 10.6 grams, after dividing the 2 grams by 12.3 and multiplying by 65 kilograms for a 65-kilogram human subject. The amount of curcumin in each IV product will be 10 mg. to one gram, and nowhere near 10.6 grams. PubChem reports curcumin as generally nontoxic, with no reported cases of overdose. In the 2014 preclinical study by Zhu et al., curcumin at a dose of 100 mg/kg was administered to mice after weight drop contusion TBI.⁶⁸ Major improvements in recovery were noted. For human equivalency dosage, 100 mg/kg divided by 12.3 is 8.13 mg/kg, times 65 for a 65-kilogram clinical subject is 528 mg/kg. Some clinical subjects may weigh 100 kilograms or more, resulting in a human equivalent dose of 813 mg. or more. This indicates that the optimal dosage of curcumin may approach 1,000 mg, which is why this amount is selected as the upper dosage parameter for curcumin in the IV composite formula. The lower dosage parameter of 10 mg is selected because the optimal TBI treatment dosage of curcumin may approach 10 mg.

Resveratrol is generally recognized as nontoxic, with no reported cases of liver toxicity, according to PubChem.gov. Ates et al. report in a 2006 study that 100 mg/kg of resveratrol significantly attenuated secondary injury from TBI in rats.⁶⁹ 100 mg/kg divided by 6.2 (the species conversion factor for rats to human) is 16.1 mg/kg. This result times 65 for a 65-kilogram human subject is 1,046 mg. Some clinical subjects may weigh considerably more than 65 kilograms, which is why 1,500 mg is selected as the upper dosage parameter for resveratrol, prior to encapsulation in liposomes, for the IV composite formula. The lower dosage parameter of 10 mg is selected because the optimal dosage of resveratrol to treat TBI may approach 10 mg.

Taurine is a nonproteinogenic amino acid that is common in many foods and is recognized as nontoxic by PubChem.gov. PubChem.gov lists taurine with an LD50 of greater than 7 grams per kilogram, which converts to human equivalent data of 74 grams for a 65-kilogram human subject (7 divided by 6.2 for rat species of the study, times 65). The upper dosage parameter for taurine of 1,000 mg is selected because this is a common therapeutic dose for taurine, and it may represent an optimal dose for treatment of TBI. The lower dosage parameter for taurine of 10 mg is selected since the optimal dosage for taurine may approach 10 mg.

Fulvic acid is nontoxic. Dai et al. conducted very thorough toxicology and genotoxicity studies of fulvic acid with rats and mice, with strong results demonstrating nontoxicity.⁷ Fulvic acid at a dosage up to 5 grams per kilogram were administered to both species for 60 days. No toxicity or genotoxicity was found for either species The upper dosage parameter for fulvic acid of 2,000 mg is selected since this is a high yet still common dosage for this compound, and the optimal dose for treating TBI may approach this parameter. The lower dosage parameter of 10 mg for fulvic acid was chosen since the optimal dosage for fulvic acid may approach 10 mg.

Acetyl L-carnitine is an endogenous substance and is therefore nontoxic in proper amounts. According to PubChem.gov, the LD50 for intravenous acetyl L-carnitine is 1,265 mg/kg for rat species, which translates to a human equivalent dosage of 13.2 grams (1,265 mg divided by 6.2, times 65 for a 65-kilogram human patient). One possible dosage for acetyl L-carnitine would be 500 mg, which would ensure general nontoxicity.

### REFERENCES

1. https://www.cdc.gov/mmwr/volumes/66/ss/ss6609a1.htm
2. “Estimating the global incidence of traumatic brain injury”, Dewan et al., 2018, https://thejns.org/view/journals/j-neurosurg/130/4/article-p1080.xml
3. “Traumatic brain injury: integrated approaches to improve prevention, clinical care, and research”, Maas et al., 2017, https://www.thelancet.com/journals/laneur/article/PIIS1474-4422(17)30371-X/fulltext
4. https://www.mayoclinic.org/diseases-conditions/traumatic-brain-injury/diagnosis-treatment/drc-20378561
5. “Guidelines for the management of severe TBI—4^{th} edition”, Brain Trauma Foundation, 2016, https://braintrauma.org/coma/guidelines/guidelines-for-the-management-of-severe-tbi-4th-ed
6. “Treatment of traumatic brain injury with anti-inflammatory drugs”, Bergold, 2016, https://www.ncbi.nlm.nih.gov/pmc/articles/PMC6007860/
7. “Neuronal degeneration and iNOS expression in experimental brain contusion following treatment with colchicine, dexamethasone, tirilazad mesylate, and nimodipine”, Gahm et al., 2005, https://pubmed.ncbi.nlm.nih.gov/16044358/
8. “Propofol induces nuclear localization of Nrf2 under conditions of oxidative stress in cardiac H9c2 cells”, Shinjo et al., 2018, https://www.ncbi.nlm.nih.gov/pmc/articles/PMC5915683/
9. “Are GABAergic drugs beneficial in providing neuroprotection after traumatic brain injuries? A comprehensive literature review of preclinical studies”, Sudhakar, 2023, https://www.frontiersin.org/articles/10.3389/fneur.2023.1109406/full
10. “Propofol is an allosteric agonist with multiple binding sites on concatemeric ternary GABA_{A} receptors”, Shin et al., 2018, https://pubmed.ncbi.nlm.nih.gov/29192122/
11. “Chloride dysregulation, seizures, and cerebral edema: a relationship with therapeutic potential”, Glykys et al., 2017, https://pubmed.ncbi.nlm.nih.gov/28431741/
12. “Emerging research and clinical development trends of liposome and lipid nanoparticle drug delivery systems”, Kraft et al., 2014, https://www.ncbi.nlm.nih.gov/pmc/articles/PMC4074410/pdf/nihms-577214.pdf
13. “Multi-dose intravenous administration of neutral and cationic liposomes in mice: an extensive toxicity study”, Andrade et al., 2022, https://www.ncbi.nlm.nih.gov/pmc/articles/PMC9229811/
14. https://www.gmp-compliance.org/files/guidemgr/Asept_Process_5882fnl.pdf
15. “Massive efflux of adenosine triphosphate into the extracellular space immediately after experimental traumatic brain injury”, Moro et al., 2021, https://www.ncbi.nlm.nih.gov/pmc/articles/PMC8027727/
16. “Intracarotid administration of liposomal-entrapped ATP: improved efficiency against experimental brain ischemia”, Laham et al., 1988, https://pubmed.ncbi.nlm.nih.gov/3212008/
17. “Liposome-delivered ATP effectively protects the retina against ischemia-reperfusion injury”, Dvoriantchikova et al., 2010, https://www.ncbi.nlm.nih.gov/pmc/articles/PMC3013064/
18. “ATP-loaded liposomes for treatment of myocardial ischemia”, Hartner et al., 2009, https://pubmed.ncbi.nlm.nih.gov/20049815/
19. “ATP-loaded liposomes effectively protect mechanical functions of the myocardium from global ischemia in an isolated rat heart model”, Verma et al., 2005, https://www.ncbi.nlm.nih.gov/pmc/articles/PMC1634739/
20. “Detecting adenosine triphosphate in the pericellular space”, Falzoni et al., 2013, https://royalsocietypublishing.org/doi/10.1098/rsfs.2012.0101
21. “Cost-effective production of ATP and S-adenosylmethionine using engineered multidomain scaffold proteins”, Yan et al., 2021, https://www.ncbi.nlm.nih.gov/pmc/articles/PMC8616028/
22. “Protection-free one-pot synthesis of 2′-deoxynucleoside 5′-triphosphates and DNA polymerization”, Caton-Williams et al., 2011, https://www.ncbi.nlm.nih.gov/pmc/articles/PMC3163101/
23. https://my.clevelandclinic.org/health/diseases/22831-vitamin-b12-deficiency
24. “Vitamin B12 enhances nerve repair and improves functional recovery after traumatic brain injury by inhibiting ER-stress induced neuron injury”, Wu et al., 2019, Frontiers in Pharmacology, https://www.frontiersin.org/articles/10.3389/fphar.2019.00406/full
25. “Developing a single-stage continuous process strategy for vitamin B12 production with Propionibacterium freudenreichii”, Calvillo et al., 2023, https://microbialcellfactories.biomedcentral.com/articles/10.1186/s12934-023-02029-x
26. “Vitamin B12 catalyzed reactions”, Giedyk et al., 2015, https://pubmed.ncbi.nlm.nih.gov/25945462/
27. “Oxidative stress and modification of synaptic proteins in hippocampus after traumatic brain injury”, Ansari et al., 2008, https://www.ncbi.nlm.nih.gov/pmc/articles/PMC2586827/
28. “Isolation of indigenous glutathione producing Saccharomyces cerevisiae strains”, Tahmasebi et al., 2016, https://www.ncbi.nlm.nih.gov/pmc/articles/PMC5563933/
29. “Extraction and concentration of glutathione from yeast by membranes”, Wang et al., 2021, https://onlinelibrary.wiley.com/doi/abs/10.1002/cjce.24084
30. https://files labcorp.com/testmenu-d8/sample_reports/007700.pdf
31. “Curcumin attenuates cerebral edema following traumatic brain injury in mice: a possible role for aquaporin-4”. Laird et al., 2010. Journal of Neurochemistry, https://www.ncbi.nlm.nih.gov/pmc/articles/PMC2911034/
32. “Curcumin suppresses inflammatory response in traumatic brain injury via p38/MAPK signaling pathway”, Li et al., 2022, Phytotherapy Research, https://onlinelibrary.wiley.com/doi/abs/10.1002/ptr.7391
33. “Curcumin attenuates acute inflammatory injury by inhibiting the TLR4/MyD88/NF-kB signaling pathway in experimental traumatic brain injury”, Zhu et al., 2014, Journal of Neuroinflammation, https://jneuroinflammation.biomedcentral.com/articles/10.1186/1742-2094-11-59
34. “Curcumin mitigates axonal injury and neuronal cell apoptosis through the PERK/Nrf2 signaling pathway following diffuse axonal injury”, Huang et al., 2018, NeuroReport, https://journals.lww.com/neuroreport/fulltext/2018/06010/curcumin_mitigates_axonal_injury_and_neuronal_cell.9.aspx
35. “Supplements, nutrition, and alternative therapies for the treatment of traumatic brain injury”, Lucke-Wold et al., 2018, https://www.ncbi.nlm.nih.gov/pmc/articles/PMC5491366/
36. “Turmeric extract supplementation reduces tau protein level in repetitive traumatic brain injury model”, Siahaan et al., 2018, https://www.ncbi.nlm.nih.gov/pmc/articles/PMC6290398/
37. “The chemistry of curcumin: from extraction to therapeutic agent”, Priyadarsini et al., 2014, https://www.ncbi.nlm.nih.gov/pmc/articles/PMC6270789/pdf/molecules-19-20091.pdf
38. “Supercritical fluid extraction with CO2 of curcuma longa L. in comparison to conventional solvent extraction”, Widmann et al., 2022, https://www.ncbi.nlm.nih.gov/pmc/articles/PMC9502963/pdf/pharmaceutics-14-01943.pdf
39. “Inhibitory effect of curcumin, a food spice from turmeric, on platelet-activating factor- and arachidonic acid-mediated platelet aggregation through inhibition of thromboxane formation and Ca2+ signaling”, Shah et al., 1999, https://pubmed.ncbi.nlm.nih.gov/10484074/
40. “Regulatory effects of curcumin on platelets: an update and future directions”, Hussain et al., 2022, https://www.mdpi.com/2227-9059/10/12/3180
41. “Curcumin at low doses potentiates and at high doses inhibits ABT-737-induced platelet apoptosis”, Rukoyatkina et al., 2021, https://www.mdpi.com/1422-0067/22/10/5405
42. “Characterization of platelet dysfunction after trauma”, Kutcher et al., 2012, https://www.ncbi.nlm.nih.gov/pmc/articles/PMC3387387/
43. https://www.labcorp.com/tests/501620/aspirinworks-11-dehydro-thromboxane-b2
44. “Resveratrol attenuates cognitive deficits in traumatic brain injury by activating p38 signaling in the brain”. Shi et al., 2018, Medical Science Monitor, https://www.ncbi.nlm.nih.gov/pmc/articles/PMC5830922/
45. “Neuroprotection by resveratrol against traumatic brain injury in rats”, 2006. Ates et al, https://link.springer.com/article/10.1007/s11010-006-9253-0
46. “Resveratrol as a therapeutic choice for traumatic brain injury: an insight into its molecular mechanism of action”, Nath et al., 2022, Brain Disorders. https://www.sciencedirect.com/science/article/pii/S2666459322000099
47. “Neuroprotective effects of resveratrol against traumatic brain injury in rats: involvement of synaptic proteins and neuronal autophagy”, Feng et al., 2016, Molecular Medicine Reports, https://www.spandidos-publications.com/10.3892/mmr.2016.5201
48. “Resveratrol attenuates early brain injury (EBI) after experimental subarachnoid hemorrhage via inhibition of NLRP3 inflammasome activation”, Zhang et al., 2017, Frontiers in Neuroscience, https://www.frontiersin.org/articles/10.3389/fnins.2017.00611/full
49. “Resveratrol protects astrocytes against traumatic brain injury through inhibiting apoptotic and autophagic cell death”, Lin et al., 2014, Cell Death & Disease, https://www.nature.com/articles/cddis2014123
50. “Resveratrol attenuates behavioral impairments and reduces cortical and hippocampal loss in a rat controlled cortical impact model of traumatic brain injury”, Singleton et al., 2010, Journal of Neurotrauma, https://www.liebertpub.com/doi/full/10.1089/neu.2010.1291
51. “Resveratrol reduces brain injury after subarachnoid hemorrhage by inhibiting oxidative stress and endoplasmic reticulum stress”, Xie et al., 2019, Neural Regeneration Research, https://journals.lww.com/nrronline/Fulltext/2019/14100/Resveratrol_reduces_brain_injury_after.20.aspx
52. “Resveratrol attenuates endoplasmic reticulum stress-induced cell death and results in functional improvement after traumatic brain injury in mice”, Nyanzu et al., 2022, SCIREA Journal of Biology, https://www.scirea.org/journal/PaperInformation?PaperID=7173
53. “A simple method for the isolation and purification of resveratrol from Polygonum cuspidatum”. Wang et al ., 2012, https://www.sciencedirect.com/science/article/pii/S2095177912001426
54. “The genoprotective activity of resveratrol on permethrin-induced genotoxic damage in cultured human lymphocytes”, Turkez et al., 2013, https://www.scielo.br/j/babt/a/Q8DDhHDd3x3jny7pDwmMypn/?lang=en
55. “The red wine phenolics trans-resveratrol and quercetin block human platelet aggregation and eicosanoid synthesis: implications for protection against coronary heart disease”, Pace-Asciak et al., 1995, https://pubmed.ncbi.nlm.nih.gov/7554275/
56. “Antiplatelet activity of synthetic and natural resveratrol in red wine”, Bertelli et al., 1995, https://pubmed.ncbi.nlm.nih.gov/7499059/
57. “Protective effects of taurine against inflammation, apoptosis, and oxidative stress in brain injury”, Niu et al., 2018, https://www.ncbi.nlm.nih.gov/pmc/articles/PMC6172387/
58. “Taurine and its analogs in neurological disorders: focus on therapeutic potential and molecular mechanisms”, Jakaria et al., 2019, https://www.sciencedirect.com/science/article/pii/S2213231719301971
59. “Taurine and astrocytes: a homeostatic and neuroprotective relationship”, Ramirez-Guerrero et al., 2022, https://www.frontiersin.org/articles/10.3389/fnmol.2022.937789/full
60. “Continuous process for the production of taurine from monoethanolamine”, Ma et al., 2020 https://pubs.acs.org/doi/pdf/10.1021/acs.iecr.0c02277
61. “A comprehensive toxicological assessment of fulvic acid”, Dai et al., 2020, https://www.ncbi.nlm.nih.gov/pmc/articles/PMC7758121/
62. “Acetyl L-carnitine in Alzheimer disease: a short-term study on CSF neurotransmitters and neuropeptides”, Bruno et al., 1995, https://pubmed.ncbi.nlm.nih.gov/8534410/
63. “Basic methods for preparation of liposomes and studying their interactions with different compounds, with the emphasis on polyphenols”, Sturm et al., 2021, https://www.ncbi.nlm.nih.gov/pmc/articles/PMC8234105/
64. “Liposome-delivered ATP effectively protects the retina against ischemia-reperfusion injury”, Dvoriantchikova et al., 2010, https://www.ncbi.nlm.nih.gov/pmc/articles/PMC3013064/
65. “A simple practice guide for dose conversion between animals and human”, Nair et al., 2016, https://www.ncbi.nlm.nih.gov/pmc/articles/PMC4804402/
66. “Adenosine-5′-triphosphate (ATP) supplementation improves low peak muscle torque and torque fatigue during repeated high intensity exercise sets”, Rathmacher et al., 2012, https://pubmed.ncbi.nlm.nih.gov/23046855/
67. https://www.mayoclinic.org/drugs-supplements-vitamin-b12/art-20363663
68. “Curcumin attenuates acute inflammatory injury by inhibiting the TLR4/MyD88/NF-kB signaling pathway in experimental traumatic brain injury”, Zhu et al., 2014, Journal of Neuroinflammation, https://jneuroinflammation.biomedcentral.com/articles/10.1186/1742-2094-11-59
69. “Neuroprotection by resveratrol against traumatic brain injury in rats”, 2006. Ates et al, https://link.springer.com/article/10.1007/s11010-006-9253-0
70. “A comprehensive toxicological assessment of fulvic acid”, Dai et al., 2020, https://www.ncbi.nlm.nih.gov/pmc/articles/PMC7758121/

### OTHER REFERENCES

71. “Management of traumatic brain injury patients”, Dash et al., 2018, https://www.ncbi.nlm.nih.gov/pmc/articles/PMC5809702/
72. “Electrospun nanofiber sheets incorporating methylcobalamin promote nerve regeneration and functional recovery in a rat sciatic nerve crush injury model”, Suzuki et al., 2017, https://pubmed.ncbi.nlm.nih.gov/28179161/
73. “Methylcobalamin increases Erk ½ and Akt activities through the methylation cycle and promotes nerve regeneration in a rat sciatic nerve injury model”, Okada et al., 2010, https://pubmed.ncbi.nlm.nih.gov/20045411/
74. “Methylcobalamin promotes the differentiation of Schwann cells and remyelination in lysophosphatidylcholine-induced demyelination of the rat sciatic nerve”, Nishimoto et al., 2015, https://www.frontiersin.org/articles/10.3389/fncel.2015.00298/full
75. “Amelioration of acute sequelae of blast-induced mild traumatic brain injury by N-acetyl cysteine: a double-blind, placebo controlled study”, Hoffer et al., 2013, https://www.ncbi.nlm.nih.gov/pmc/articles/PMC3553161/
76. “Anti-oxidant therapies in traumatic brain injury”, Pietro et al., 2020. Antioxidants, https://www.ncbi.nlm.nih.gov/pmc/articles/PMC7139349/
77. “The role of serial oxidative stress levels in acute traumatic brain injury and as predictors of outcome”, Wang, et al., 2016, Science Direct, World Neurosurgery, https://pubmed.ncbi.nlm.nih.gov/26481337/
78. “Glutathione precursors shield the brain from trauma”, Koza, Linseman, 2019, Neural Regeneration Research, https://www.ncbi.nlm.nih.gov/pmc/articles/PMC6585556/
79. “Platelet dysfunction in the perioperative and critical care setting”, Ho et al., 2018, https://jeccm.amegroups.com/article/view/4346/4950
80. “Administration of riboflavin improves behavioral outcome and reduces edema formation and glial fibrillary acidic protein expression after traumatic brain injury”, Hoane et al., 2005, Journal of Neurotrauma, https://pubmed.ncbi.nlm.nih.gov/16238487/
81. “Thiamine preserves mitochondrial function in a rat model of traumatic brain injury, preventing inactivation of the 2-oxoglutarate dehydrogenase complex”. Mrktchyan et al., 2018, https://pubmed.ncbi.nlm.nih.gov/29777685/
82. “Treatment with Vitamin B3 improves functional recovery and reduces GFAP expression following traumatic brain injury in rats”, Hoane et al., 2003, Journal of Neurotrauma, https://pubmed.ncbi.nlm.nih.gov/14651806/
83. “The use of nicotinamide as a treatment for experimental traumatic brain injury and stroke: a review and evaluation”, Vonder Haar et al., 2013, https://tbitherapy.com/use-nicotinamide-treatment-experimental-traumatic-brain-injury-stroke-review-evaluation/
84. “In vivo detection of extracellular adenosine triphosphate in a mouse model of traumatic brain injury”, Faroqi et al., 2021, https://www.ncbi.nlm.nih.gov/pmc/articles/PMC7898407/
85. “Extracellular ATP and neurodegeneration”, Bernardi, 2003, https://www.eurekaselect.com/article/36603 (Robert Chittenden's comment: ATP only adds to neuropathology if excess ATP has migrated to the brain extracellular space where it binds with purinergic receptors, thus signaling for increased neuroinflammation. If the ATP is delivered into neuron membranes, which may be accomplished by liposomes, evidence from studies indicates that ATP in this physiological context leads to decreases in pathological levels of neuroinflammation.)
86. “Trovafloxacin attenuates neuroinflammation and improves outcome after traumatic brain injury in mice”, Garg et al., 2019, https://pubmed.ncbi.nlm.nih.gov/29439712/(Robert Chittenden's comment: Many studies indicate researchers are thinking in terms of reducing ATP signaling after TBI instead of increasing intracellular ATP without increasing extracellular ATP. Thus, many researchers are not considering ATP in a liposomal context, which appears to be one of the few ways to augment intraneuronal ATP without directly increasing extracellular ATP.)
87. “Oral polyunsaturated phosphatidylcholine reduces platelet lipid and cholesterol contents in healthy volunteers”. Galli et al., 1985, https://pubmed.ncbi.nlm.nih.gov/3900615/
88. “L-Carnitine and extendin-4 improve outcomes following moderate brain contusion injury”, Chen et al., 2018, https://www.nature.com/articles/s41598-018-29430-6
89. “Clinical evidence of acetyl-L-carnitine efficacy in the treatment of acute ischemic stroke: a pilot clinical trial”, Mazdeh et al., 2022, https://www.hindawi.com/journals/omcl/2022/2493053/
90. “Brain phospholipid precursors administered post-injury reduce tissue damage and improve neurological outcome in experimental traumatic brain injury”, Thau-Zuchman et al., 2018, https://www.ncbi.nlm.nih.gov/pmc/articles/PMC6306688/(Robert Chittenden's comment: The ingredients of the invention of this study include cyanocobalamin, which is an unmethylated version of vitamin B12, which could have different metabolic effects after TBI compared to methylated B12, especially for patients with genetic deficiencies in methylation.)
91. “Review: traumatic brain injury and hyperglycemia, a potentially modifiable risk factor”, Shi et al., 2016, https://www.ncbi.nlm.nih.gov/pmc/articles/PMC5342608/
92. “Brain hypoxia and ischemia after traumatic brain injury”, Vespa, 2016 https://jamanetwork.com/journals/jamaneurology/article-abstract/2506513
93. “Dynamics of choline-containing phospholipids in traumatic brain injury and associated comorbidities”, Javaid et al., 2021, https://www.ncbi.nlm.nih.gov/pmc/articles/PMC8583393/
94. “Lipid profiling of brain tissue and blood after traumatic brain injury: a review of human and experimental studies”, Nessel et al., 2021, https://www.sciencedirect.com/science/article/abs/pii/S1084952120300793
95. “Intracerebral iron accumulation may be associated with secondary brain injury in patients with poor grade subarachnoid hemorrhage”, Helbok et al., 2022, https://www.ncbi.nlm.nih.gov/pmc/articles/PMC8813702/
96. “Methods of liposomes preparation: formation and control factors of versatile nanocarriers for biomedical and nanomedicine application”, Lombardo et al., 2022, https://www.ncbi.nlm.nih.gov/pmc/articles/PMC8955843/
97. “Liposomes: advancements and innovation in the manufacturing process”, Shah et al., 2020, https://www.sciencedirect.com/science/article/pii/S0169409X20300788
98. “An introduction to liposome processing for drug delivery”, Bozenhardt et al., 2020, https://www.pharmaceuticalonline.com/doc/an-introduction-to-liposome-processing-for-drug-delivery-0001
99. “Vitamins and nutrients as primary treatments in experimental brain injury: clinical implications for nutraceutical therapies”, Vonder Haar et al., 2016, https://www.ncbi.nlm.nih.gov/pmc/articles/PMC4870112/
100. “Natural compounds as a therapeutic intervention following traumatic brain injury: the role of phytochemicals”, Scheff et al., 2017, https://www.ncbi.nlm.nih.gov/pmc/articles/PMC5749600/
101. “Therapeutic potential of fulvic acid in chronic inflammatory diseases and diabetes”, Winkler et al., 2018, https://www.ncbi.nlm.nih.gov/pmc/articles/PMC6151376/
102. “ATP-induced cellular stress and mitochondrial toxicity in cells expressing purinergic P2X7 receptor”, Seeland et al., 2015, https://www.ncbi.nlm.nih.gov/pmc/articles/PMC4448979/

### U.S. Pat. Nos.:

U.S. Pat. No. 5,547,942
U.S. Pat. No. 7,629,329
U.S. Pat. No. 8,252,325
U.S. Pat. No. 8,784,881
U.S. Pat. No. 10,272,130
U.S. Pat. No. 10,603,329

1. The product comprises a colloidal suspension that will include ingredients liposomal adenosine triphosphate salt, methylcobalamin, glutathione, liposomal curcumin, liposomal resveratrol, taurine, fulvic acid, and acetyl L-carnitine, that will be delivered intravenously or by other parenteral means to treat traumatic brain injury.

2. Product of claim #1, wherein liposomes used to encapsulate certain ingredients will generally be less than 120 nanometers in diameter, and optimally between 70 nanometers and 100 nanometers in diameter, to reduce immune system clearance and to improve delivery to damaged brain parenchyma.

3. Product of claim #1 will be further comprised of liposomal adenosine triphosphate salt at a dosage that exceeds 10 mg of adenosine triphosphate salt, where the precise dosage will provide efficacy against secondary pathology caused by traumatic brain injury, yet the precise dosage will not exceed an amount that would cause a statistically significant increase of ATP in the brain's extracellular space after a traumatic brain injury.

4. Product of claim #1, wherein glutathione will be removed from the formula for patients with a history of Stevens-Johnson syndrome or toxic epidermal necrolysis, until additional testing can determine whether glutathione contributes to symptoms of these conditions.

5. Product of claim #1 will be further comprised of liposomal curcumin at a dosage of 10 mg to 1,000 mg of curcumin before liposomal encapsulation, where the precise dosage will not exceed an amount that would produce a statistically significant and adverse decrease in platelet aggregation after a traumatic brain injury.

6. Product of claim #1 will be further comprised of liposomal resveratrol at a dosage of 10 mg to 1,500 mg of resveratrol before liposomal encapsulation, where the precise dosage will not exceed an amount that would produce a statistically significant and adverse decrease in platelet aggregation after a traumatic brain injury.

7. Product of claim #1 will be further comprised of taurine at a dosage of 10 mg to 1,000 mg, where the precise dosage will not exceed an amount that would cause a statistically significant increase of neuronal influx of chloride ions after a traumatic brain injury.

8. Product of claim #1 will be further comprised of fulvic acid at a dosage of 10 mg to 2,000 mg, where the precise dosage will not exceed an amount that would cause a statistically significant increase in cerebral free iron levels after a traumatic brain injury.
